(19) United States
(12) Patent Application Publication
(10) Pub. No.: US 2011/0190612 A1
(43) Pub. Date: Aug. 4, 2011

(54) Continous Light Emission Photoacoustic Spectroscopy

(75) Inventors: Edward M. McKenna, Boulder, CO (US); Youzhi Li, Longmont, CO (US)

(73) Assignee: Nellcor Puritan Bennett LLC, Boulder, CO (US)

(21) Appl. No.: 13/019,742

(22) Filed: Feb. 2, 2011

Related U.S. Application Data
(60) Provisional application No. 61/300,756, filed on Feb. 2, 2010.

(51) Int. Cl. A61B 6/00; A61B 5/1455

(52) U.S. Cl. 600/323; 600/476

(57) ABSTRACT

Methods and systems are provided for analyzing microcirculation using photoacoustic spectroscopy by emitting continuous light at one or more frequencies. A photoacoustic spectroscopy monitor may utilize a slow modulation method to vary the wavelength of light emitted, such that different absorbers may be measured in a patient's tissue. The photoacoustic spectroscopy sensor may emit a lower power continuous light towards a patient's tissue. The acoustic response generated by the tissue may be sensed by a thin polymer sensing film at the detector of the sensor. Based on the amplitude and phase information of the acoustic response sensed by the detector, the monitor may determine a concentration of an absorber, as well as a location of the absorbers, in the patient's tissue.

### RELATED APPLICATION

This application claims the benefit of U.S. Provisional Application No. 61/300,756, filed Feb. 2, 2010, which application is hereby incorporated by reference.

### BACKGROUND

The present disclosure relates generally to medical devices and, more particularly, to the use of continuous light emission in photoacoustic spectroscopy to analyze microcirculation.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, doctors often desire to monitor certain physiological characteristics of their patients. Accordingly, a wide variety of devices have been developed for monitoring many such physiological characteristics. Such devices provide doctors and other healthcare personnel with the information they need to provide the best possible healthcare for their patients. As a result, such monitoring devices have become an indispensable part of modern medicine.

For example, clinicians may wish to monitor a patient's blood flow and blood oxygen saturation to assess cardiac function. Deviation from normal or expected values may alert a clinician to the presence of a particular clinical condition. A patient's microcirculatory system, which includes the arterioles and capillaries, is involved in delivering blood to various tissues and organs, and changes in blood delivery to these tissues or organs may be an indication of injury or disease. Thus, by monitoring changes in microcirculation, a clinician may be able to diagnose or monitor diseases in particular organs or tissues. In addition, changes in microcirculation may predict systemic changes that present earlier or more profound microcirculatory changes, followed by changes in blood flow to larger vessels. For example, in cases of shock or pathogenic infection, a clinical response may include shunting of blood from the micro circulatory system to the larger vessels in an attempt to increase blood flow and prevent injury to primary organs (e.g., the brain and heart) while temporarily decreasing blood flow to secondary organs (e.g., the gastrointestinal system or the skin).

Microcirculation may be analyzed using techniques for assessing blood volume. Some techniques may be invasive and involve the use of radioisotopes or other tagged blood indicators. The indicators may be tracked through the circulation to estimate the blood volume. Many of these techniques involve indirect assessment of blood volume by measuring the density or concentration of certain blood constituents. For example, sound velocity measurements may be used for measuring several hemodynamic parameters. However, such sensors utilize a linear approximation of a non-linear relationship between the sound velocity and the density of the blood. This approximation may limit the accuracy of the technique. In addition, these techniques may not be suitable for assessing local changes in microcirculation.

### BRIEF DESCRIPTION OF THE DRAWINGS

Advantages of the disclosed techniques may become apparent upon reading the following detailed description and upon reference to the drawings in which:

FIG. 1 illustrates a simplified block diagram of a pulse oximeter, according to an embodiment; and

FIG. 2 is a flow chart depicting a process for modulating a continuous wave source to emit varying frequencies into a patient's tissue.

### DETAILED DESCRIPTION OF SPECIFIC EMBODIMENTS

One or more specific embodiments of the present techniques will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Present embodiments relate to noninvasively monitoring microcirculation in a patient using photoacoustic spectroscopy. Monitoring a patient's microcirculation may involve estimating the patient's total blood volume or changes in the patient's total blood volume, which may provide information about the patient's clinical condition. Further, changes in microcirculation may be seen first in the smallest vessels, e.g., arterioles, capillaries, and so forth. Therefore, a photoacoustic spectroscopy sensor suitable for analyzing such small vessels may provide information to allow earlier monitoring and/or detection of microcirculatory changes. In some embodiments, microcirculation monitoring may allow clinicians to diagnose patients based at least in part on changes in microcirculation. Sensors as provided may be applied to a patient's skin or mucosal tissue to monitor the microcirculatory parameters. For example, such sensors may be suitable for use on any area of a patient with a sufficient density of microcirculatory vessels. Such areas include digits, ears, cheeks, the lingual and sublingual area, and/or the upper respiratory tract (e.g., the esophagus, trachea or lungs, which may be accessible through tracheostomy tubes or endotracheal tubes). In one embodiment, such sensors may be used to monitor organ tissue that may be accessible while a patient is undergoing surgical treatment.

Photoacoustic spectroscopy involves a light source suitable for emitting light into a tissue such that the emitted light is absorbed by certain components of the tissue and/or blood. The absorption of the light energy results in the generation of kinetic energy, which results in pressure fluctuations at the measurement site. The pressure fluctuations may be detected in the form of acoustic radiation (e.g., ultrasound). As different absorbers and concentrations of absorbers at a tissue measurement site may absorb different wavelengths of light, the amplitude of the detected acoustic radiation may be correlated to a density or concentration of a particular absorber that absorbs at the wavelength of the emitted light. Furthermore, a phase difference between the detected acoustic radiation and the emitted light may indicate a position in the measurement site (e.g., a depth in the tissue relative to the photoacoustic spectroscopy sensor). Thus, by emitting a light beam at a wavelength absorbed by components in the tissue and/or blood, photoacoustic spectroscopy may be used to estimate microcirculatory blood volume, as well as other parameters, at particular measurement sites.

Photoacoustic spectroscopy may provide certain advantages for the examination of microcirculation, and an acoustic response produced in photoacoustic spectroscopy may provide additional information compared to a traditional blood monitoring device. For example, the acoustic wave detected by a photoacoustic spectroscopy sensor may generate a signal which may be proportional to the absorption spectrum of the tissue and/or blood and also indicative of a location of the absorbers being measured. More specifically, certain parameters of the acoustic wave, such as phase, amplitude, and waveform, may be analyzed to determine physiological information, such as blood-oxygen level and total hemoglobin, at various depths from the sensor. Furthermore, in some embodiments, a relatively low-power continuous wave source may be employed as a continuous light source for a photoacoustic spectroscopy sensor. In one such embodiment, a thin polymer transducer may be used to detect the pressure fluctuations associated with the acoustic wave. Utilizing a thinner polymer transducer may enable increased sensitivity and a higher signal to noise ratio, as well as greater flexibility in the design of the sensor.

Furthermore, in one embodiment, the light emitted by a sensor may be modulated at some frequency (e.g., 10 MHz to 100 MHz) and the light may be emitted at different wavelengths. Different measuring sites (e.g., digits, ears, cheeks, the lingual and sublingual area, etc.) may have various types of fluid and/or tissue which absorb different wavelengths of light. Emitting light at different wavelengths may provide different information depending on the absorption properties of the absorbers (e.g., blood, other fluids, and/or tissue, etc.) associated with a measurement site. Thus, present embodiments may involve modulating a light emitted from a photoacoustic spectroscope, such that one or more wavelengths of light may be emitted to analyze different absorbers at various measurement sites on the patient. Further, the modulation techniques may involve a slow modulation (e.g., between 100 KHz to 10 MHz), such that pressure wave changes resulting from the changes in the emitted light may be detected.

Provided herein are systems, sensors, and methods for monitoring microcirculation. Such systems may involve using a photoacoustic spectroscopy sensor in conjunction with a medical monitor, which may assess one or more parameters indicative of microcirculation, including blood volume or flow in a tissue bed perfused with microcirculatory vessels, the depth or distribution of microcirculatory vessels, or the concentration of blood constituents in the microcirculatory vessels, etc. Systems and methods of the present techniques may also involve using a continuous wave light source. The continuous wave source may be lower power than a typical pulsed wave light source, such that a thin polymer sensing film may be used in detecting an acoustic response. Further, the light source may be modulated at different frequencies such that different wavelengths of light may be transmitted.

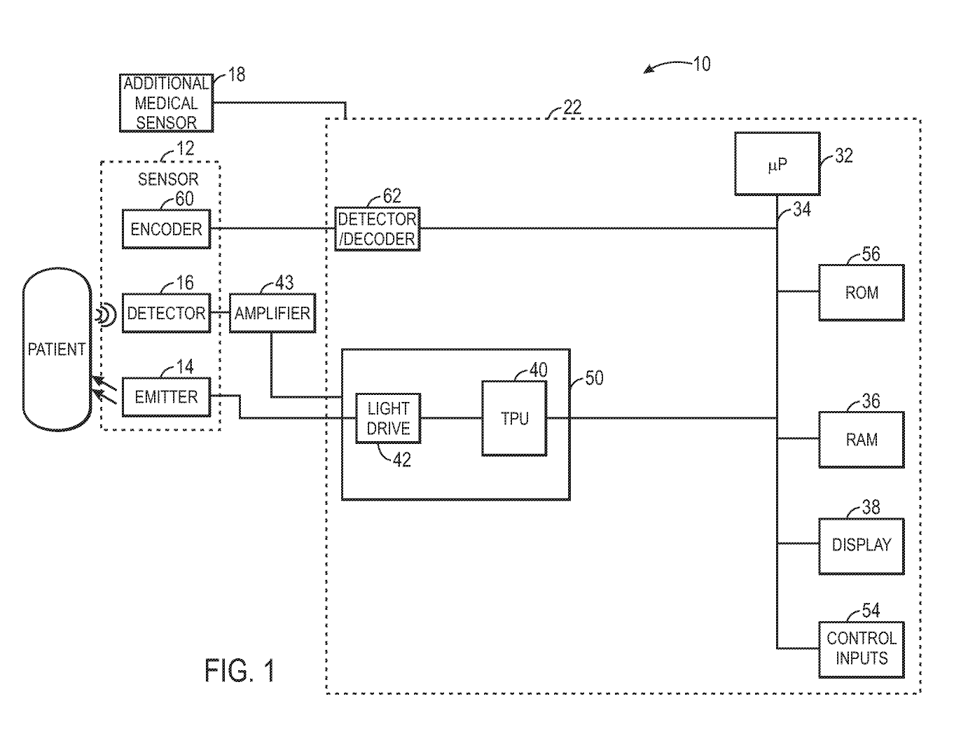

FIG. 1 shows a system 10 that may be used for monitoring microcirculation. The system 10 includes a photoacoustic spectroscopy sensor 12 with a light source 14 and acoustic detector 16. The sensor 12 may emit a light in a continuous manner when in operation, though in some embodiments, a pulsed light source may instead be used in the system 10. A pulsed wave source may typically emit higher power light in shorter pulses while a continuous wave light source may emit a lower power light continuously. A continuous wave light source may allow for longer-term monitoring of tissue, due to less heating of the tissue. Because microcirculation takes place in relatively small blood vessels, such heating may effect blood flow in the vessels and may interfere with sensor measurements. Thus, continuous wave light sources, which may result in less heating of the tissue, may provide certain advantages when used in a photoacoustic spectroscopy sensor 12 to analyze microcirculation. In one embodiment, the relatively low power of light emitted by the continuous wave source may range from approximately 50 mW to 1 W.

The sensor assembly 10 includes a light emitter 14 and an acoustic detector 16 that may be of any suitable type. The emitter 14 may include one or more light emitting diodes (LEDs) adapted to transmit one or more wavelengths of light as discussed herein, and the detector 16 may include one or more ultrasound transducers configured to receive ultrasound waves generated by the tissue in response to the emitted light and to generate a corresponding electrical or optical signal. In specific embodiments, the emitter 14 may be a laser diode or a vertical cavity surface emitting laser (VCSEL). The laser diode may be a tunable laser, such that a single diode may be tuned to various wavelengths corresponding to a number of absorbers. Depending on the particular arrangement of the photoacoustic sensor 12, the emitter 14 may be associated with an optical fiber for transmitting the emitted light into the tissue. The light may be any suitable wavelength corresponding to the wavelengths absorbed by certain constituents in the blood. For example, wavelengths between about 500 nm to about 600 nm, corresponding with green visible light, may be absorbed by deoxyhemoglobin and oxyhemoglobin. In other embodiments, red and near infrared wavelengths may be used. Further, the emitted light may be modulated at any suitable frequency, such as 1 MHz or 10 MHz.

The acoustic detector 16 may be any receiver suitable for receiving a response generated by the tissue when exposed to the emitted light. In some embodiments, the response may be a pressure fluctuation, an acoustic wave, a thermal wave, or any other non-optical wave generated by the conversion of absorbed light energy into kinetic energy. An acoustic wave will be used herein as one example of the tissue's response to the emitted light, which is detected by the detector 16. While a pulsed wave system may utilize a comparably more complex acoustic detector suitable for detecting the acoustic wave generated in response to higher powered pulsed light, an acoustic detector 16 for a continuous wave photoacoustic spectroscopy sensor may be a standard detector model suitable for detecting acoustic waves generated using lower power light emissions. Further, in one embodiment, the acoustic wave generated by a continuous light source may provide a higher signal to noise ratio relative to that generated by a pulsed light.

In one embodiment, the detector 16 may be a low finesse Fabry-Perot interferometer, which may include a thin polymer sensing film mounted at the tip of an optical fiber. The thin sensing film may allow a higher sensitivity to be achieved than a thicker sensing film. Using a Fabry-Perot polymer film interferometer, an incident acoustic wave emanating from the probed tissue may modulate the thickness of the thin polymer film and the phase difference of the light reflected from the two sides of the polymer film. The light reflection produces a corresponding intensity modulation of the light reflected from the film. Accordingly, the acoustic wave may be converted to optical information, which may be transmitted through an optical fiber to a suitable optical detector. The change in phase of the detected light may be detected via an appropriate interferometry device. The use of the thin polymer film allows high sensitivity to be achieved, even for films of micrometer or tens of micrometers in thickness. For example, in one embodiment, the thin film may be a 0.25 mm diameter disk of 50 micrometer thickness polyethylene terepthalate with an at least partially optically reflective (e.g., 40% reflective) aluminum coating on one side and a mirror reflective coating on the other (e.g., 100% reflective) that form the mirrors of the interferometer. The optical fiber may be any suitable fiber, such as a 50 micrometer core silica multimode fiber of numerical aperture 0.1 and an outer diameter of 0.25 mm.

As discussed, the continuous light emitted by the emitter 14 may be sinusoidally modulated, and the light energy emitted towards the tissue may be converted to kinetic energy when absorbed, which may generate pressure fluctuations in the tissue. The pressure fluctuations may be detected by the detector 16 as an acoustic wave, which may be proportional to the absorption of the modulated light by one or more absorbers in the tissue. The detector 16 may be coupled to a lock-in amplifier 43, which may be configured to output a voltage signal proportional to the acoustic wave generated in the tissue. The lock-in amplifier 43 may use, for example, a frequency mixer, to lock onto the frequency of the acoustic wave and convert the phase and amplitude of the acoustic wave to a voltage signal. In one embodiment, the sinusoidal modulation of the continuous wave source may be a relatively slow modulation (e.g., 100 KHz to 20 MHz) such that the lock-in amplifier 43 may lock in on the acoustic wave with higher accuracy, improving the signal to noise ratio of the output voltage response. Further, the slow modulation of the continuous wave source may also reduce the technological requirements on the lock-in amplifier 43, such that simpler and/or less costly lock-in amplifiers may be used. In embodiments, the lock-in amplifier 43 may be implemented in the sensor 12, or may be external from the sensor 12 (e.g., in the monitor 22).

The system 10 may, in embodiments, also include any number or combination of additional medical sensors 18 or sensing components for providing information related to patient parameters that may be used in conjunction with the photoacoustic spectroscopy sensor 12. For example, suitable sensors may include sensors for determining blood pressure, blood constituents (e.g., oxygen saturation), respiration rate, respiration effort, heart rate, patient temperature, or cardiac output. Such information may be used in conjunction with microcirculation information to determine a physiological condition of a patient. For example, additional medical sensors 18 may include a pulse oximetry sensor, a blood pressure cuff, or other non-invasive blood pressure sensors, and so forth. Further, in certain embodiments, a photoacoustic spectroscopy sensor 12 may be a multi-parameter sensor, such as with a unitary housing, which includes additional components for pulse oximetry sensing or other cardiac or blood constituent sensing.

The system 10 may also include a monitor 22 which may receive signals from the photoacoustic spectroscopy sensor 12 and, in some embodiments, from one or more additional sensors 18. The monitor 22 may analyze microcirculation based on the signals received by the photoacoustic spectroscopy sensor 12 and/or the one or more additional sensors 18. For example, in embodiments in which an additional sensor 18 is a pulse oximetry sensor, the pulse oximetry signal may generate a plethysmographic waveform, which may be further processed by the monitor 22. The monitor 22 may receive and process a signal from the photoacoustic spectroscopy sensor 12 to determine a physiological condition based on the signals generated by the photoacoustic spectroscopy sensor 12 and/or any additional sensors 18. In one embodiment, the monitor 22 may indicate a condition related to microcirculatory parameters (e.g., a patient's likelihood of being in sepsis) and/or indicate other information representative of a physiological condition.

The monitor 22 may include a microprocessor 32 coupled to an internal bus 34. Also connected to the bus may be a RAM memory 36 and a display 38. A time processing unit (TPU) 40 may provide timing control signals to light drive circuitry 42, which controls the emission of light by a sensor (e.g., a photoacoustic spectroscopy sensor 12 or any other additional medical sensor 18) when activated, and, if multiple light sources are used, the multiplexed timing for the different light sources. TPU 40 may also control the gating-in of signals from the sensor 12 and a switching circuit 44. These signals are sampled at the proper time, depending at least in part upon which light sources are activated, if multiple light sources are used, and/or which wavelengths of light are being emitted if the emitted light is modulated at more than one wavelength. In some embodiments, the signal received from the sensor 12 may be passed through one or more signal processing elements, such as an amplifier, a low pass filter, and/or an analog-to-digital converter. Furthermore, in some embodiments, digital data may be stored in a suitable storage component in the monitor 22, for example, in a queued serial module, RAM 36, or ROM 56.

The TPU 40 and the light drive circuitry 42 may be part of a modulator 50, which may modulate the drive signals from the light drive circuitry 42 that activate the LEDs or other emitting structures of the emitter 14. The modulator 50 may be hardware-based, software-based, or some combination thereof. For example, a software aspect of the modulator 50 may be stored on the memory 36 and may be executed by the processor 32. In some embodiments, the modulator 50 may be configured to modulate a continuous wave light emitted from the photoacoustic spectroscopy sensor 12, and may be any modulator suitable for modulating a continuous wave source at a low power. If a pulsed wave were to be emitted, the modulator 50 may be suitable for modulating higher power pulses of light. While the modulator 50 is depicted as in the monitor 22, in some embodiments, the modulation function may be performed by a modulator disposed in the photoacoustic spectroscopy sensor 12. In one embodiment, the modulation and detection features may both be located within the sensor(s) 12 and/or 18 to reduce the distance traveled by the signals, and to reduce potential interferences.

In an embodiment, based at least in part upon the signals generated by the detector 16 in response to the detected acoustic waves, microprocessor 32 may calculate the microcirculation parameters using various algorithms. Patient conditions may be analyzed based on signals received from the sensor 12 and, in embodiments, other sensors 18 (e.g., pulse oximetry sensor), and/or control inputs 54 input by a user. For example, a caregiver may input a patient's age, weight, gender, or information about the patient's clinical condition that may be relevant in analyzing patient conditions. These algorithms used to calculate the microcirculation parameters may employ certain coefficients, which may be empirically determined, and may correspond to the wavelength of light used. In addition, the algorithms may employ additional correction coefficients. The algorithms and coefficients may be stored in a ROM 56 or other suitable computer-readable storage medium and accessed and operated according to microprocessor 32 instructions. In one embodiment, the correction coefficients may be provided as a lookup table. In addition, the sensor 12 may include certain data storage elements, such as an encoder 60, that may encode information related to the characteristics of the sensor 12, including information about the emitter 14 and/or the detector 16. The information may be accessed by detector/decoder 62, located on the monitor 22.

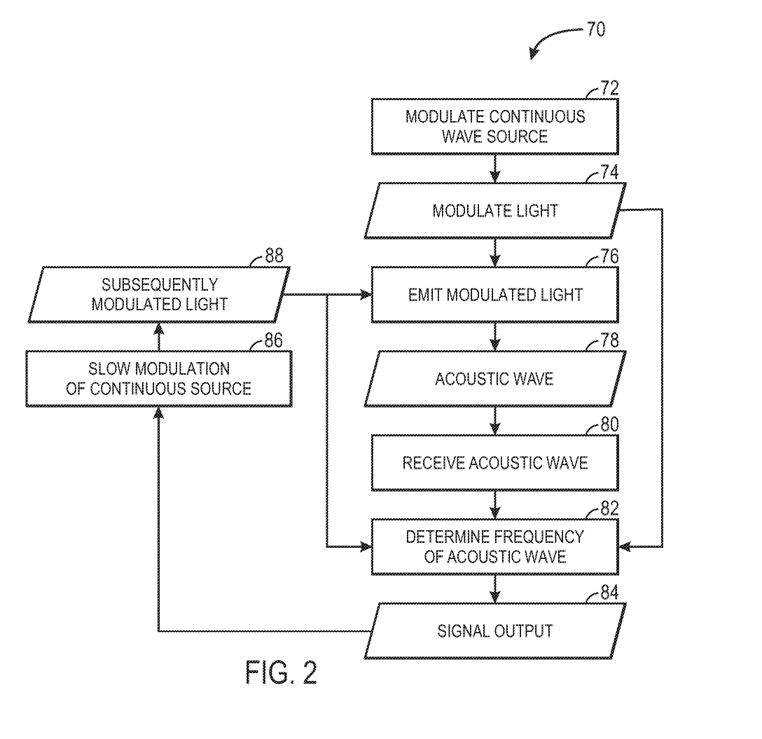

One embodiment of a process 70 for modulating a continuous light source of a photoacoustic spectroscopy sensor to emit light at variable wavelengths is provided as a flow chart in FIG. 2. The process 70 may include modulating (block 72) a light source of a sensor 12 (as in FIG. 1) to emit a modulated beam 74 at a suitable wavelength. The modulation process may involve controlling the operating current of, for example, a laser diode in the emitter 14, which may be substantially controlled by light drive circuitry 42 in a modulator 50 of a photoacoustic spectroscopy system 10. The modulation frequency may be based on various factors, including the physiological conditions of a patient being measured, the measurement site on the patient, the absorbers of interest at the measurement site, the conditions to be analyzed, and/or the photoacoustic spectroscopy system limitations. For example, if the patient is being measured sublingually, the emitted light may be modulated to wavelengths between about 500 nm to about 600 nm, corresponding with green visible light, which may be absorbed by oxyhemoglobin in the oral cavity.

Once the sensor 12 emits (block 76) the modulated light towards the patient's tissue, the light energy may be absorbed by certain components of the tissue (e.g., absorbers) based on the wavelength of the light, the concentration and/or amount of the absorbers at the measurement site, and/or the absorption coefficients of the absorbers. The absorbed light energy may be converted to kinetic energy, which generates an acoustic response 78 in the tissue at the measurement site. The acoustic wave 78 may be received (block 80) by a detector 16 in the photoacoustic spectroscopy sensor 12. The detector 16 may also be coupled to a lock-in amplifier 43 which locks-in the frequency (block 82) of the acoustic wave 78. The amplifier 43 may lock into a frequency of the acoustic wave 78 based on a frequency of the modulated light 74. Thus, the lock-in amplifier 43 may perform a phase-sensitive detection process by determining the amplitude of the acoustic wave 78, as well as the phase shift between the acoustic wave 78 and the waveform of the emitted modulated light 74.

Based on a comparison of the waveform of the emitted modulated light 74 and the detected acoustic wave 78, the amplitude component of the acoustic wave 78 and the phase shift between the acoustic wave 78 and the emitted modulated light 74 may provide information as to the concentration and/or location of the absorbers being measured. The amplitude component of the acoustic wave 78 may provide information corresponding to the concentration of absorbers being measured, as the intensity of the acoustic reaction 78 may be proportional to the amount of light absorbed by the absorbers having a certain absorption coefficient. The phase component of the acoustic wave 78 may provide information corresponding to the location of the absorbers being measured. More specifically, the phase component may be a time delay between the modulated light 74 and the acoustic response 78. The monitor 22 may determine (e.g., based on algorithms executed by a microprocessor 32) that the sensor 12 is measuring absorbers at a certain depth in the tissue based on the phase information of the acoustic reaction 78. The lock-in amplifier 43 may output a voltage signal 84 of the amplitude and phase information of the acoustic wave 78. As discussed, this signal 84 may be used by the monitor 22 for further processing and/or analyses of microcirculation at the measurement site.

In one embodiment, a slow modulation process may be implemented to vary the frequency of the emitted light. Once an acoustic response is locked in at a frequency, the process 70 may involve slowly modulating (block 86) the light source to emit a subsequently modulated beam 88 having a different frequency than the previously emitted modulated light 76. Varying the frequency of the light source may enable greater flexibility and/or produce more information regarding the concentrations of different absorbers at different measuring sites. For example, modulating the light source may result in emitting light at a different wavelength, which may be absorbed by different absorbers (which may have different absorption coefficients) at a measuring site, thus producing more information on concentrations of one or more absorbers at the tissue being measured. Further, as some absorbers of interest may have different absorption coefficients depending on the measuring site (e.g., a digit, the oral cavity, etc.), emitting light at different wavelengths may enable measurements of absorbers of interest at various measuring sites on the patient. In embodiments, one wavelength of light may be emitted by a sensor 12 at one time or at different times. Further, in an embodiment, a sensor 12 may have more than one light source, such that more than one wavelength of light may be emitted at once.

A signal corresponding to the waveform of the subsequently modulated light 88 may be fed back to the detector 16 and/or the lock-in amplifier 23, such that the acoustic response 78 may be analyzed with respect to the frequency, amplitude, and phase of the subsequently emitted light 88. Phase shifts between the subsequently modulated light 88 and the acoustic response 78 generated by the subsequently modulated light 88 may also be sensed and output 84 by the lock-in amplifier 43. Further, the modulation may be slow, such that changes in the acoustic waves 78 in response to changes in the emitted light 74 and 88 may be detected and locked in by the amplifier 43.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Rather, the various embodiments may cover all modifications, equivalents, and alternatives falling within the spirit and scope of the disclosure as defined by the following appended claims.

What is claimed is:

1. A method, comprising:
modulating a continuous light source in a photoacoustic spectroscopy sensor to emit a light having a wavelength absorbable by an absorber in a patient's tissue;
emitting the modulated light towards the patient's tissue; and
determining an amplitude component and a phase component of an acoustic wave generated in response to the emitted modulated light.

2. The method, as set forth in claim 1, wherein modulating the continuous light source comprises modulating the continuous light source at two or more frequencies.

3. The method, as set forth in claim 2, wherein the two or more frequencies are based on one or more of a physiological condition of the patient, one or more absorbers in the patient's tissue, or a configuration of the photoacoustic spectroscopy sensor.

4. The method, as set forth in claim 1, wherein the continuous light source emits light continuously, at approximately 50 mW to 1 W.

5. The method, as set forth in claim 1, wherein the amplitude component and the phase component of the acoustic wave are determined based on a comparison of the acoustic wave with a waveform of the emitted modulated light.

6. The method, as set forth in claim 1, wherein the amplitude component of the acoustic wave indicates a concentration of the absorber.

7. The method, as set forth in claim 1, wherein the phase component of the acoustic wave indicates a location of the absorber in the patient's tissue.

8. A photoacoustic spectroscopy system, comprising:
a continuous wave light source configured to be modulated to emit one or more wavelengths of light into a patient's tissue;
a detector configured to receive a response wave generated in the patient's tissue in response to the light emitted by the continuous wave light source, wherein the response wave is non-optical; and
a processor configured to determine a concentration of an absorber in the patient's tissue based on the response wave and the one or more wavelengths of light.

9. The system, as set forth in claim 8, comprising a modulator configured to modulate the continuous wave light source through a range of frequencies.

10. The system, as set forth in claim 8, wherein the response wave is one or more of a pressure wave, an acoustic wave, or a thermal wave.

11. The system, as set forth in claim 8, wherein the detector comprises a Fabry-Perot polymer film transducer.

12. The system, as set forth in claim 8, wherein the detector comprises a film between about 1 μm to about 50 μm in thickness.

13. The system, as set forth in claim 8, comprising a lock-in amplifier configured to determine a frequency of the response wave based on the light emitted into the patient's tissue.

14. The system, as set forth in claim 13, wherein the lock-in amplifier is configured to output a voltage signal comprising one or more of amplitude information or phase information of the response wave.

15. The system, as set forth in claim 8, comprising a photoacoustic spectroscopy sensor configured to continuously emit the one or more wavelengths of light, and further configured to receive the response wave.

16. The system, as set forth in claim 8, further comprising a pulse oximetry sensor configured to emit one or more wavelengths of light to the patient's tissue and receive the light that has been transmitted through or scattered by the patient's tissue.

17. The system, as set forth in claim 8, comprising memory storing algorithms directed to calculating the concentration of the absorber and the depth of the absorber, wherein the processor is capable of accessing the memory to execute the algorithms.

18. A photoacoustic spectroscopy monitor, comprising:
a modulator configured to modulate a continuous light source;
data processing circuitry configured to receive a response to an emission of the continuous light source and determine an amplitude and a phase of the response, wherein the response comprises non-optical data; and
a processor configured to utilize one or more of the amplitude or the phase to calculate one or more of a concentration of an absorber in a patient's tissue or a location of the absorber.

19. The monitor of claim 18, wherein the response is on or more of a pressure wave, an acoustic wave, or a thermal wave.

20. The monitor of claim 18, wherein the data processing circuitry is configured to lock in a response frequency based on an emission frequency of the continuous light source.

21. The monitor of claim 18, wherein the data processing circuitry is configured to process a signal comprising the amplitude and the phase of the response.

22. The monitor of claim 18, wherein the location of the absorber is a depth in the patient's tissue of where the concentration of the absorber is determined.

23. The monitor of claim 18, wherein the absorber comprises one or more of blood, other fluids, a tissue, or any other component in the patient capable of absorbing light energy and generating a kinetic response.
